(19) United States
(12) Patent Application Publication
(10) Pub. No.: US 2024/0384210 A1
(43) Pub. Date: Nov. 21, 2024

(54) CULTURING SYSTEM

(71) Applicant: HONDA MOTOR CO., LTD., Tokyo (JP)

(72) Inventors: Shohei Kinoshita, Wako-shi (JP); Nozomi Shiobara, Wako-shi (JP); Kenji Machida, Wako-shi (JP); Minoru Goto, Wako-shi (JP); Mizuho Doi, Wako-shi (JP); Satoshi Shiozaki, Wako-shi (JP); Kensuke Takahashi, Wako-shi (JP); Ken Harada, Wako-shi (JP); Yu Arakawa, Wako-shi (JP); Hiroshi Tamura, Tokyo (JP)

(21) Appl. No.: 18/663,119

(22) Filed: May 14, 2024

(30) Foreign Application Priority Data
May 17, 2023 (JP) 2023-081351

(51) Int. Cl. C12M 1/00

(52) U.S. Cl. CPC C12M 23/58; C12M 23/40; C12M 29/26

(57) ABSTRACT

A culturing system is equipped with a culturing tank and a supply device for supplying a culturing solution to the culturing tank. The culturing tank includes a plurality of accommodation sections capable of accommodating the culturing solution. The supply device includes a liquid delivery device for delivering the culturing solution to the plurality of accommodation sections, and supply pipes for supplying the culturing solution to the plurality of accommodation sections, respectively. The culturing system includes first float valves that can open and close the supply pipes. The first float valves each include a first float.

### CROSS-REFERENCE TO RELATED APPLICATIONS

This application is based upon and claims the benefit of priority from Japanese Patent Application No. 2023-081351 filed on May 17, 2023, the contents of which are incorporated herein by reference.

### BACKGROUND OF THE INVENTION

### Field of the Invention

The present invention relates to a culturing system for culturing microalgae.

### Description of the Related Art

Heretofore, efforts aimed at mitigating or reducing the impact of climate change have continued, and toward the realization thereof, research and development in relation to the reduction of carbon dioxide emissions are being carried out. From this point of view, attention has been focused on microalgae. This is because microalgae consume carbon dioxide through photosynthesis. Accordingly, a culturing system for culturing microalgae is anticipated as a system that contributes to alleviating or reducing the impact of climate change.

The culturing system includes a tank for storing a culturing solution, an accommodation section for accommodating the culturing solution and microalgae and culturing the microalgae, and a control unit. The tank and the accommodation section are connected to each other through a supply pipe. The supply pipe is provided with a solenoid valve. When the culturing solution is supplied from the tank to the accommodation section, the water level of the culturing solution in the accommodation section is detected by a water level sensor (liquid level indication controller) described as a conventional technique in JP H01-247077 A. The control unit acquires information relating to the water level of the culturing solution in the accommodation section. When the water level of the culturing solution reaches the water level input in advance to the control unit, the control unit transmits a control signal for closing the solenoid valve.

In the case where the culturing system includes a plurality of accommodation sections, the above-described configuration requires the same number of water level sensors and solenoid valves as the number of accommodation sections. Therefore, capital investments in equipment expenditures are increased. Further, since a cable for electrically connecting the water level sensor and the control unit and a cable for electrically connecting the solenoid valve and the control unit are provided, wiring of both cables is complicated.

In order to eliminate the need for a water level sensor and a solenoid valve, it is conceivable to provide a U-shaped pipe rotatably on the accommodation section as proposed in JP H01-247077 A. According to JP H01-247077 A, the water level of the liquid accommodated in the accommodation section can be changed without using a water level sensor, on the basis of changing an inclination angle with respect to the vertical direction by rotating the U-shaped pipe.

### SUMMARY OF THE INVENTION

In the technique described in JP H01-247077 A, it is necessary to change the inclination angle of the U-shaped pipe by manual operation of an operator. In this case, the operation is cumbersome.

The present invention has the object of solving the aforementioned problem.

According to an aspect of the present invention, a culturing system is provided. The culturing system includes a culturing tank configured to culture microalgae, a supply device configured to supply a culturing solution to the culturing tank, wherein the culturing tank includes a plurality of accommodation sections configured to accommodate the culturing solution, and the supply device includes a liquid delivery device configured to deliver the culturing solution to the plurality of accommodation sections, and supply pipes through which the culturing solution delivered from the liquid delivery device is delivered to the plurality of accommodation sections, respectively, and first float valves configured to open and close the supply pipes, wherein first floats constituting the respective first float valves close the supply pipes by moving upward in a depth direction of the plurality of accommodation sections as liquid surfaces of the culturing solution rise in the depth direction.

According to the present invention, the supply of the culturing solution to the accommodation sections can be automatically stopped when the supply pipes are closed by the first floats moving upward. Accordingly, in the culturing system, a water level sensor for detecting the liquid level of the culturing solution is not necessary. Accordingly, it is possible to reduce the cost of investment in equipment.

The above and other objects, features, and advantages of the present invention will become more apparent from the following description when taken in conjunction with the accompanying drawings, in which a preferred embodiment of the present invention is shown by way of illustrative example.

### BRIEF DESCRIPTION OF THE DRAWINGS

FIG. 1 is a schematic configuration diagram of a culturing system according to an embodiment of the present invention;

FIG. 2 is a schematic configuration diagram showing a state in which all of a plurality of accommodation sections constituting the culturing tank are empty;

FIG. 3 is a schematic configuration diagram showing a state in which the supply of the culturing solution to the accommodation section is started from the state shown in FIG. 2;

FIG. 4 is a schematic configuration diagram showing a state in which the supply of the culturing solution is completed in the accommodation section at the most upstream position from the state shown in FIG. 3;

FIG. 5 is a schematic configuration diagram showing a state in which the recovery of the culturing solution in the accommodation section is started from the state shown in FIG. 1;

FIG. 6 is a schematic configuration diagram showing a state in which the recovery of the culturing solution is completed in the accommodation section at the most upstream position from the state shown in FIG. 5; and

FIG. 7 is a schematic configuration diagram of a culturing system in which a plurality of accommodation sections are directly connected to a single liquid delivery device.

### DETAILED DESCRIPTION OF THE INVENTION

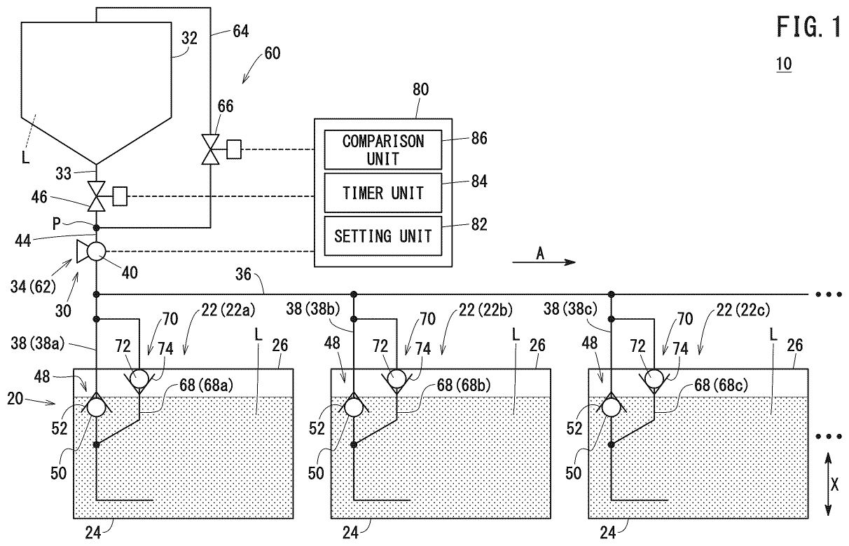

FIG. 1 is a schematic configuration diagram of a culturing system 10 according to the present embodiment. In this case, the culturing system 10 includes a culturing tank 20, a supply device 30, and a recovery device 60. The culturing tank 20 has a plurality of accommodation sections 22. In FIG. 1, three accommodation sections 22 are shown representatively. In the following description, in order to easily distinguish the three accommodation sections 22, the left accommodation section 22 in FIG. 1 is referred to as a first accommodation section 22a. Further, the central accommodation section 22 in FIG. 1 is referred to as a second accommodation section 22b, and the right accommodation section 22 in FIG. 1 is referred to as a third accommodation section 22c.

However, the configuration shown in FIG. 1 is merely an example. The number of the accommodation sections 22 may be four or more. That is, one or more accommodation sections 22 may be further provided on the right side in FIG. 1. Alternatively, the culturing tank 20 may be constituted by two of the first accommodation section 22a and the second accommodation section 22b. However, in the following description, only the first accommodation section 22a to the third accommodation section 22c will be described in order to simplify the description and facilitate its understanding.

Each of the first accommodation section 22a to the third accommodation section 22c is made of a material exhibiting flexibility. A typical example of a flexible material is linear low density polyethylene (LLDPE). Each of the first accommodation section 22a to the third accommodation section 22c is, for example, a bag-shaped object. The first accommodation section 22a to the third accommodation section 22c are retained by a retaining frame (not shown) at a predetermined installation location. When the recovery device 60 is not provided, the material of each of the first accommodation section 22a to the third accommodation section 22c need not necessarily be a material exhibiting flexibility. In this case, the material may be, for example, plastic or glass.

The direction from a bottom portion 24 to a top portion 26 of the accommodation section 22 or the direction from the top portion 26 to the bottom portion 24, is defined as a depth direction X. The depth direction X of the accommodation section 22 substantially coincides with, for example, the direction of gravity. In this case, an inclination angle of the accommodation section 22 in the depth direction X with respect to the direction of gravity is substantially zero. That is, the accommodation section 22 is in an upright posture. The depth direction X of the accommodation section 22 may intersect the direction of gravity at a predetermined angle. In such a case, the accommodation section 22 is in an inclined posture.

In a typical example, the volumes of the first accommodation section 22a to the third accommodation section 22c are substantially equal to each other. Each of the first accommodation section 22a to the third accommodation section 22c has the same depth direction X. However, the volumes of the first accommodation section 22a to the third accommodation section 22c may be different from each other, and the depth directions X of the first accommodation section 22a to the third accommodation section 22c may also be different from each other.

The supply device 30 includes a storage tank 32, an outward pipe 33, a bidirectional flow pipe 44, a liquid delivery device 34, a base pipe 36, and a supply pipe 38. Culturing solution L is stored in the storage tank 32. A typical example of the culturing solution L is water. The culturing solution L may contain nitrogen, phosphorus, potassium, and other nutrients.

In the present embodiment, the liquid delivery device 34 is constituted by a bidirectional pump 40. As will be described later, the bidirectional pump 40 serves as both of a liquid delivery device 34 constituting the supply device 30 and a suction device 62 constituting the recovery device 60.

One end of the outward pipe 33 is connected to the bottom portion of the storage tank 32. Another end of the outward pipe 33 is connected to a bidirectional pump 40 via a bidirectional flow pipe 44. One end of a return pipe 64 is connected to a connection point P between the bidirectional flow pipe 44 and the outward pipe 33. In other words, the outward pipe 33 and the return pipe 64 branch at one end of the bidirectional flow pipe 44. Another end of the return pipe 64 is routed to the upper portion of the storage tank 32 and connected to the storage tank 32 at the upper portion. The outward pipe 33 is provided with a supply valve 46. The return pipe 64 is provided with a recovery valve 66.

One end of the base pipe 36 is connected to the liquid delivery device 34. Another end of the base pipe 36 extends toward the culturing tank 20. The supply pipe 38 branches from the base pipe 36 and goes to each of the first accommodation section 22a to the third accommodation section 22c. In the following description, the supply pipe 38 extending from the base pipe 36 toward the first accommodation section 22a is referred to as a first supply pipe 38a. The supply pipe 38 extending from the base pipe 36 toward the second accommodation section 22b is referred to as a second supply pipe 38b, and the supply pipe 38 extending from the base pipe 36 toward the third accommodation section 22c is referred to as a third supply pipe 38c.

The first supply pipe 38a branches from the base pipe 36 and is inserted into the first accommodation section 22a from a top portion 26. The second supply pipe 38b branches from the base pipe 36 at a portion different from the first supply pipe 38a and is inserted into the second accommodation section 22b from a top portion 26. The third supply pipe 38c branches from the base pipe 36 at a portion different from the first supply pipe 38a and the second supply pipe 38b, and is inserted into the third accommodation section 22c from a top portion 26.

When the culturing solution L is supplied from the storage tank 32 to the culturing tank 20, a flow direction of the culturing solution L in the base pipe 36 is a direction of arrow A in FIG. 1. Accordingly, the first accommodation section 22a to the third accommodation section 22c are arranged in a manner so that they are arranged from the upstream position to the downstream position in the flow direction of the culturing solution L in the base pipe 36 when the culturing solution L is supplied. That is, the first accommodation section 22a is located at the most upstream position, the third accommodation section 22c is located at the most downstream position, and the second accommodation section 22b is located at the middle position. Hereinafter, the configuration in which the first accommodation section 22a to the third accommodation section 22c are arranged from the upstream position to the downstream position in the flow direction of the culturing solution L and sequentially connected to the base pipe 36 may be referred to as a “series arrangement (or arranged in series)”.

A first float valve 48 is provided in each of the first supply pipe 38a to the third supply pipe 38c. The first float valve 48 has a first float 50 and a first valve seat 52. The specific gravity of the first float 50 is smaller than the specific gravity of the culturing solution L. When the culturing solution L is water, the specific weight of the first float 50 is smaller than the specific gravity of water. Accordingly, when the culturing solution L is accommodated in the first accommodation section 22a to the third accommodation section 22c, the first float 50 is positioned on the liquid surface of the culturing solution L. When the first float 50 is raised to the maximum, the first float 50 comes into abutment against the first valve seat 52. The first float valve 48 is closed by this abutment. Accordingly, each of the first supply pipe 38a to the third supply pipe 38c is closed by the first float valve 48.

On the other hand, when the first float 50 is separated from the first valve seat 52, the first float valve 48 is in the open state. In this case, the culturing solution L can flow through each of the first supply pipe 38a to the third supply pipe 38c.

The first float valve 48 which opens and closes as described above functions as a check valve. Specifically, the first float valve 48 allows the culturing solution L to flow from the base pipe 36 toward the first accommodation section 22a in the first supply pipe 38a. On the other hand, the first float valve 48 prevents the culturing solution L from flowing from the first accommodation section 22a toward the base pipe 36 in the first supply pipe 38a. The same applies to the first float valve 48 provided in the second supply pipe 38b and the first float valve 48 provided in the third supply pipe 38c.

The recovery device 60 includes a recovery pipe 68, the base pipe 36, the suction device 62, the bidirectional flow pipe 44, the return pipe 64, and the storage tank 32. As described above, the suction device 62 is constituted by the bidirectional pump 40, and also serves as the liquid delivery device 34 constituting the supply device 30. Accordingly, the base pipe 36, the suction device 62 (the bidirectional pump 40), and the storage tank 32 constitute the supply device 30 and constitute the recovery device 60. The supply device 30 and the recovery device 60 may be independent devices. In this case, for example, the liquid delivery device 34 can be configured by a one-way pump, and the suction device 62 can be configured by another one-way pump.

The recovery pipe 68 branches from each of the first supply pipe 38a to the third supply pipe 38c. The recovery pipe 68 branched from the first supply pipe 38a is hereinafter referred to as a first recovery pipe 68a. Similarly, a recovery pipe 68 branched from the second supply pipe 38b is referred to as a second recovery pipe 68b, and a recovery pipe 68 branched from the third supply pipe 38c is referred to as a third recovery pipe 68c. The first recovery pipe 68a is guided so as to bypass the first float valve 48 and joins the first supply pipe 38a. Similarly, the second recovery pipe 68b and the third recovery pipe 68c are also guided so as to bypass the first float valves 48 and join the second supply pipe 38b and the third supply pipe 38c, respectively.

It is not always necessary to branch the first recovery pipe 68a to the third recovery pipe 68c from the first supply pipe 38a to the third supply pipe 38c, respectively. The first supply pipe 38a to the third supply pipe 38c and the first recovery pipe 68a to the third recovery pipe 68c may be separate pipes connected to the base pipe 36 independently of each other.

A second float valve 70 is provided in each of the first recovery pipe 68a to the third recovery pipe 68c. The second float valve 70 has a second float 72 and a second valve seat 74. The second float 72 is disposed above (higher than) the first float 50. When one of the first floats 50 is raised to the maximum and closes one of the first supply pipe 38a to the third supply pipe 38c, the second float 72 is positioned above the liquid surface of the culturing solution L in a state in which the second float 72 abuts against the second valve seat 74. In this case, the second float valve 70 is in the closed state.

The second floats 72 are separated from the second valve seats 74 when the culturing solution L in the first accommodation section 22a to the third accommodation section 22c is sucked by the suction device 62 (the bidirectional pump 40). In this case, each of the second float valves 70 is in an open state.

The second float valve 70 which opens and closes as described above functions as a check valve. Specifically, the second float valve 70 prevents the culturing solution L from flowing from the base pipe 36 toward the first accommodation section 22a in the first recovery pipe 68a. On the other hand, the second float valve 70 allows the culturing solution L to flow from the first accommodation section 22a toward the base pipe 36 in the first recovery pipe 68a. The same applies to the second float valve 70 provided in the second recovery pipe 68b and the second float valve 70 provided in the third supply pipe 38c.

A part of the first supply pipe 38a and a part of the first recovery pipe 68a are exposed from the first accommodation section 22a. Similarly, a part of the second supply pipe 38b and a part of the second recovery pipe 68b are exposed from the second accommodation section 22b, and a part of the third supply pipe 38c and a part of the third recovery pipe 68c are exposed from the third accommodation section 22c. It is not essential to seal the top portion 26 of each of the first accommodation section 22a to the third accommodation section 22c, but the top portion 26 may be sealed.

The culturing system 10 further includes a control unit 80. The control unit 80 may be configured by a processor such as a central processing unit (CPU). More specifically, the control unit 80 is configured by a processing circuitry. In this case, the processor executes computer-executable instructions stored in an unillustrated memory. The control unit 80 is electrically connected to the supply valve 46, the recovery valve 66, and the bidirectional pump 40.

The control unit 80 includes a setting unit 82, a timer unit 84, and a comparison unit 86. A required time period from when the supply of the culturing solution L to the empty first accommodation section 22a to the empty third accommodation section 22c is started until when all the first float valves 48 provided in the first supply pipe 38a to the third supply pipe 38c are placed in the closed state, is set in the setting unit 82. The required time period can be obtained by performing a preliminary test. Further, a pressure threshold value relating to the discharge pressure of the bidirectional pump 40 is set in the setting unit 82.

The timer unit 84 measures an actual elapsed time period from the start of the supply of the culturing solution L to the empty first accommodation section 22a to the empty third accommodation section 22c. The comparison unit 86 compares the actual elapsed time period with the required time period set in the setting unit 82. Alternatively, the comparison unit 86 acquires information on an actual discharge pressure of the bidirectional pump 40, and compares the actual discharge pressure with the pressure threshold value set in the setting unit 82.

The culturing system 10 according to the present embodiment is basically configured in the manner described above. Next, the operation state of the culturing system 10 when the culturing solution L is supplied to the culturing tank 20 will be described.

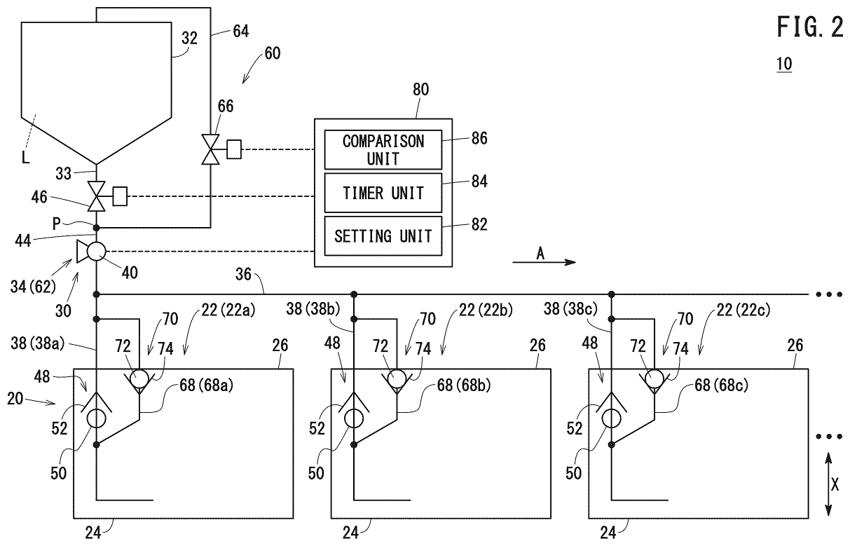

FIG. 2 is a schematic configuration diagram showing a state before the culturing solution L is supplied to the culturing tank 20. FIG. 2 shows a case where the bidirectional pump 40 is stopped and the supply valves 46 and the recovery valves 66 are each in a closed state. In this state, the culturing solution L in the storage tank 32 is prevented from flowing through the bidirectional flow pipe 44. Accordingly, the culturing solution L is also prevented from being supplied from the storage tank 32 to the first accommodation section 22a to the third accommodation section 22c. Therefore, the first accommodation section 22a to the third accommodation section 22c are empty. In each of the first accommodation section 22a to the third accommodation section 22c, the first float valve 48 is in an open state in which the first float 50 is separated from the first valve seat 52, and the second float valve 70 is in a closed state in which the second float 72 abuts against the second valve seat 74. The second float 72 is lowered by gravity and comes into abutment against the second valve seat 74.

In the case that the culturing solution L is supplied from the storage tank 32 to the first accommodation section 22a to the third accommodation section 22c, the operator instructs the control unit 80 to “start the supply of the culturing solution L to the first accommodation section 22a to the third accommodation section 22c” via an input device (not shown). For example, the operator turns a supply start switch ON. As a result, the control unit 80 places the supply valve 46 in an open state, and drives the bidirectional pump 40. In this case, the bidirectional pump 40 functions as the liquid delivery device 34, and delivers the culturing solution L from the bidirectional flow pipe 44 toward the base pipe 36. As a result, the culturing solution L flows into the base pipe 36 through the outward pipe 33, the bidirectional flow pipe 44, and the bidirectional pump 40. The timer unit 84 of the control unit 80 starts measuring the actual elapsed time from the start of the liquid delivery.

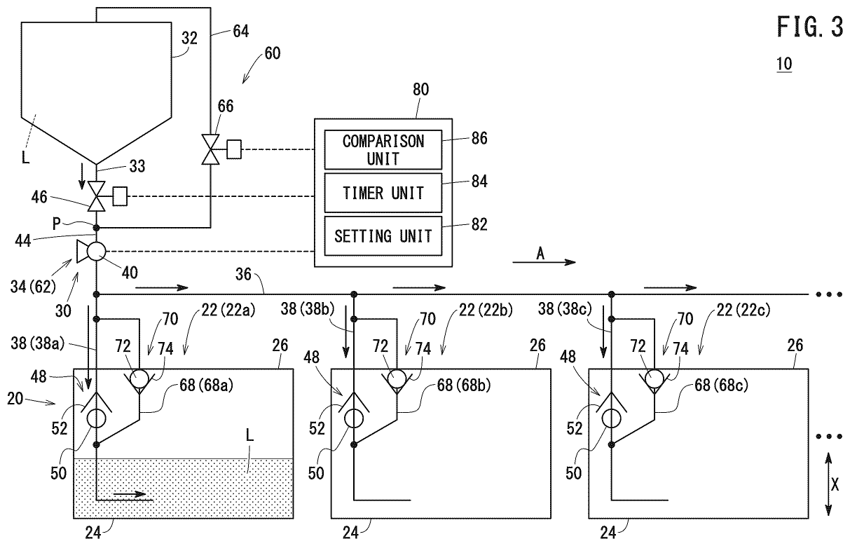

When the culturing solution L flows in the base pipe 36 in the direction of arrow A shown in FIGS. 1 and 2, a part of the culturing solution L flows into the first supply pipe 38a. Since the first float valve 48 provided in the first supply pipe 38a is in the open state, the culturing solution L passes through the first float valve 48 and is discharged into the first accommodation section 22a as shown in FIG. 3. Since the second float valve 70 is in the closed state, the culturing solution L is prevented from being supplied to the first accommodation section 22a via the first recovery pipe 68a.

A part of the culturing solution L which does not flow into the first supply pipe 38a but further flows in the direction of arrow A in the base pipe 36 flows into the second supply pipe 38b. Since the first float valve 48 provided in the second supply pipe 38b is in the open state, the culturing solution L passes through the first float valve 48 and is discharged into the second accommodation section 22b. Since the second float valve 70 is in the closed state, the culturing solution L is prevented from being supplied to the second accommodation section 22b via the second recovery pipe 68b.

A part of the culturing solution L that does not flow into the second supply pipe 38b but flows further in the direction of arrow A in the base pipe 36 flows into the third supply pipe 38c. Since the first float valve 48 provided in the third supply pipe 38c is in the open state, the culturing solution L passes through the first float valve 48 and is discharged into the third accommodation section 22c. Since the second float valve 70 is in the closed state, the culturing solution L is prevented from being supplied to the third accommodation section 22c via the third recovery pipe 68c.

In the present embodiment in which the first accommodation section 22a to the third accommodation section 22c are arranged in series in order from the upstream position in the flow direction (direction of arrow A) of the culturing solution L, when all the first float valves 48 are in the open state, the culturing solution L is supplied preferentially in the order of the first accommodation section 22a located at the most upstream position, the second accommodation section 22b located at the midstream position, and the third accommodation section 22c located at the most downstream position. That is, the inflow amount of the culturing solution L per unit time in the first accommodation section 22a is larger than the inflow amount of the culturing solution L per unit time in the second accommodation section 22b. The inflow amount of the culturing solution L per unit time in the second accommodation section 22b is larger than the inflow amount of the culturing solution L per unit time in the third accommodation section 22c.

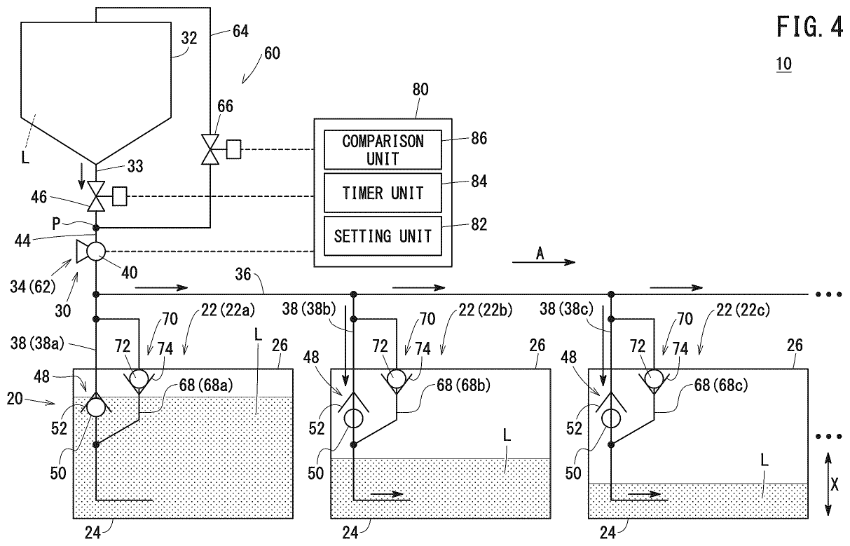

For the above reason, as shown in FIGS. 3 and 4, the liquid level of the culturing solution L rises in the first accommodation section 22a before the liquid level in the second accommodation section 22b. Similarly, the liquid level of the culturing solution L rises in the second accommodation section 22b before the liquid level in the third accommodation section 22c.

Accordingly, as shown in FIG. 4, the first float 50 is lifted and seated on the first valve seat 52 in the first float valve 48 of the first accommodation section 22a in a shorter time than the first float valve 48 of the second accommodation section 22b and the first float valve 48 of the third accommodation section 22c. That is, the first float valve 48 of the first accommodation section 22a is placed in the closed state. Accordingly, the supply of the culturing solution L to the first accommodation section 22a is stopped. In contrast, the first float valve 48 of the second accommodation section 22b and the first float valve 48 of the third accommodation section 22c are still in the open state. Therefore, the supply of the culturing solution L to the second accommodation section 22b and the third accommodation section 22c are continued.

For the above reason, the liquid level of the culturing solution L rises in the second accommodation portion 22b before the liquid level in the third accommodation portion 22c. Accordingly, the first float valve 48 of the second accommodation section 22b is closed in a shorter time than the first float valve 48 of the third accommodation section 22c. Accordingly, the supply of the culturing solution L to the second accommodation section 22b is stopped. In contrast, since the first float valve 48 of the third accommodation section 22c is still in the open state, the supply of the culturing solution L to the third accommodation section 22c is continued.

Next, the liquid level of the culturing solution L rises in the third accommodation section 22c, and the first float valve 48 is closed. As a result, the supply of the culturing solution L to the third accommodation section 22c is stopped, and the state shown in FIG. 1 is obtained. Consequently, the first supply pipe 38a to the third supply pipe 38c are switched from the open state to the closed state.

In each of the first accommodation section 22a to the third accommodation section 22c, the second float valve 70 is positioned above (higher than) the first float valve 48. Accordingly, when the culturing solution L is supplied to the first accommodation section 22a to the third accommodation section 22c, all the second float valves 70 are kept in the closed state. Therefore, the culturing solution L is prevented from being supplied to the first accommodation section 22a to the third accommodation section 22c via the first recovery pipe 68a to the third recovery pipe 68c.

In the preliminary test, the time required from when the supply of the culturing solution L to the culturing tank 20 is started until when the first float valve 48 of the third accommodation section 22c is placed in the closed state, is measured. The required time period is set in the setting unit 82 in advance. The comparison unit 86 compares the required time period set in the setting unit 82 with the actual elapsed time period measured by the timer unit 84. When the required time period and the actual elapsed time period coincide with each other, the control unit 80 determines that “a predetermined amount of the culturing solution L is accommodated in each of the first accommodation section 22a to the third accommodation section 22c”. Next, the control unit 80 stops the bidirectional pump 40.

After all the first float valves 48 are closed, the actual discharge pressure of the bidirectional pump 40 increases. The comparison unit 86 may compare the pressure threshold value set in the setting unit 82 with the actual discharge pressure of the bidirectional pump 40. When the actual discharge pressure reaches the pressure threshold value, the control unit 80 will determine that “a predetermined amount of the culturing solution L is accommodated in each of the first accommodation section 22a to the third accommodation section 22c”. Next, the control unit 80 stops the bidirectional pump 40.

Both the comparison between the required time period and the actual elapsed time period, and the comparison between the pressure threshold value and the actual discharge pressure, may be performed simultaneously. In such a case, the control unit 80 stops the bidirectional pump 40 when either the actual elapsed time period coincides with the required time period or the actual discharge pressure reaches the pressure threshold value. By promptly stopping the bidirectional pump 40 after the supply of the culturing solution L to the culturing tank 20 is completed as mentioned, an excessive load is prevented from acting on the bidirectional pump 40. The control unit 80 switches the supply valve 46 to the closed state.

After the culturing solution L is contained in the culturing tank 20, the microalgae are cultured in the culturing tank 20. Although not particularly shown, gas is supplied to the culturing tank 20 and light is irradiated to the culturing tank 20 during culturing.

After the culturing of the microalgae is completed, the culturing solution L containing the microalgae is recovered from the culturing tank 20 as follows. First, in the state shown in FIG. 1, the operator instructs the control unit 80 to “start the recovery of the culturing solution L from the first accommodation section 22a to the third accommodation section 22c” through the input device. For example, the operator turns a recovery start switch ON. As a result, the control unit 80 places the recovery valve 66 in an open state, and drives the bidirectional pump 40. In this case, the bidirectional pump 40 functions as the suction device 62, and suctions the culturing solution L through the first supply pipe 38a to the third supply pipe 38c.

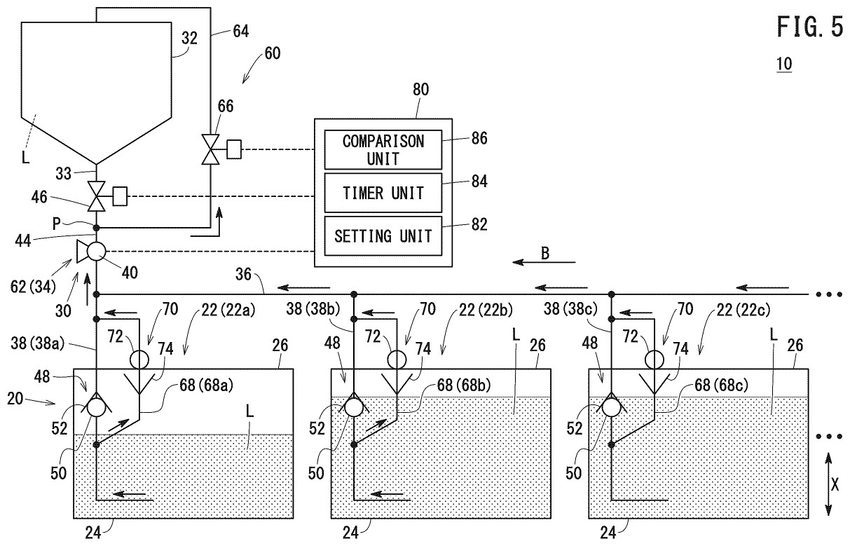

In each of the first supply pipe 38a to the third supply pipe 38c, the first float 50 of the first float valve 48 has already been raised and abuts against the first valve seat 52. The direction in which the culturing solution L is suctioned from the culturing tank 20 is the direction in which the first float 50 is raised. Therefore, the first float 50 is kept in the state in which the first float 50 abuts against the first valve seat 52 as shown in FIG. 5 while the culturing solution L is sucked from the culturing tank 20. That is, the first float valve 48 maintains the closed state. Accordingly, the culturing solution L is prevented from passing through the first float valve 48.

On the other hand, the direction in which the culturing solution L is suctioned from the culturing tank 20 is the direction in which the second float 72 is raised from the second valve seat 74. Accordingly, in each of the first recovery pipe 68a to the third recovery pipe 68c, as shown in FIG. 5, the second float 72 is separated from the second valve seat 74, and the second float valve 70 is placed in the open state. Accordingly, the first recovery pipe 68a to the third recovery pipe 68c are switched from the closed state to the open state. As a result, the interior of the first accommodation section 22a to the third accommodation section 22c and the base pipe 36 communicate with each other through the first recovery pipe 68a to the third recovery pipe 68c, respectively.

Accordingly, the culturing solution L in the first accommodation section 22a to the third accommodation section 22c is suctioned. The culturing solution L flows through the base pipe 36 in the direction of arrow B in FIG. 5, and then flows into the storage tank 32 through the bidirectional flow pipe 44 and the return pipe 64. As a result, the culturing solution L is recovered in the storage tank 32 in a state in which the culturing solution L contains the microalgae.

The first accommodation section 22a is in the closest proximity to the bidirectional pump 40 among the first accommodation section 22a to the third accommodation section 22c that are arranged in series. Therefore, in the case that all the first float valves 48 are in the closed state, the culturing solution L is preferentially suctioned in the order of the first accommodation section 22a, the second accommodation section 22b, and the third accommodation section 22c. That is, the amount of the culturing solution L suctioned into the first accommodation section 22a per unit time is larger than the amount of the culturing solution L suctioned into the second accommodation section 22b per unit time. The amount of the culturing solution L suctioned into the second accommodation section 22b per unit time is larger than the amount of the culturing solution L suctioned into the third accommodation section 22c per unit time.

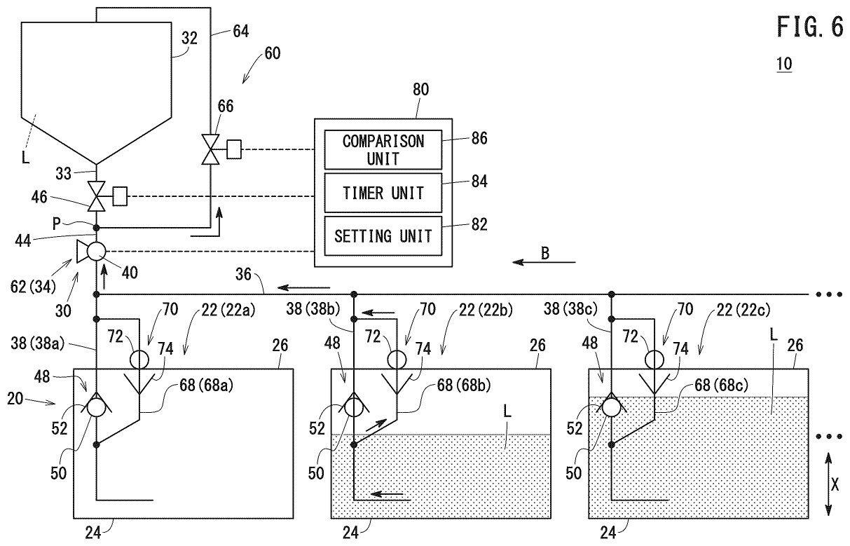

For the above reason, as shown in FIGS. 5 and 6, the suction of the culturing solution L is finished in the first accommodation section 22a before the suction in the second accommodation section 22b. Similarly, the suction of the culturing solution L is finished in the second accommodation section 22b before the suction in the third accommodation section 22c.

As described above, in the present embodiment, the material of the first accommodation section 22a to the third accommodation section 22c exhibits flexibility. Therefore, as the recovery of the culturing solution L from the first accommodation section 22a to the third accommodation section 22c progresses, the first accommodation section 22a to the third accommodation section 22c are pressed and crushed by the atmosphere. That is, the first accommodation section 22a to the third accommodation section 22c are contracted. Therefore, for example, when the suction of the culturing solution L has been completed in the first accommodation section 22a as shown in FIG. 6, the inside of the first accommodation section 22a is in a vacuum state (at a negative pressure). All the first float valves 48 maintain the closed state. For the above reason, the culturing solution L is prevented from returning from the first recovery pipe 68a to the third recovery pipe 68c to the first accommodation section 22a to the third accommodation section 22c, respectively, even though all the second float valves 70 are kept in the open state.

In this situation, the culturing solution L remaining in the second accommodation section 22b is recovered in preference to the culturing solution L remaining in the third accommodation section 22c. When the suction of the culturing solution L has been completed in the second accommodation section 22b, the inside of the second accommodation section 22b is in a vacuum state. Therefore, the second accommodation section 22b is pressed by the atmosphere and is crushed (contracted) in a state that the second float valve 70 in the second recovery pipe 68b is kept in an open state.

In this situation, the culturing solution L remaining in the third accommodation section 22c is recovered. When the suction of the culturing solution L has been completed in the third accommodation section 22c, the inside of the third accommodation section 22c is in a vacuum state. Therefore, the second accommodation section 22b is pressed by the atmosphere and is collapsed (contracted) in a state where the second float valve 70 in the third recovery pipe 68c is kept in an open state. The recovery of the culturing solution L from the culturing tank 20 is thus completed.

In the preliminary test, the required time period from when the recovery of the culturing solution L from the culturing tank 20 is started until when the third accommodation section 22c is placed in a vacuum state may be measured, and the required time period may be set in the setting unit 82. In this case, the comparison unit 86 compares the required time period set in the setting unit 82 with the actual elapsed time period from when the recovery of the culturing solution L from the culturing tank 20 is started. When the required time period and the actual elapsed time period coincide with each other, the control unit 80 determines that “the collection of the culturing solution L from the first accommodation section 22a to the third accommodation section 22c has been completed”. Next, the control unit 80 stops the bidirectional pump 40.

Further, a pressure threshold value relating to the suction pressure of the bidirectional pump 40 may be set in the setting unit 82. In this case, the comparison unit 86 compares the pressure threshold value set in the setting unit 82 with the actual suction pressure of the bidirectional pump 40. In the case that the actual suction pressure reaches the pressure threshold value, the control unit 80 determines that “the collection of the culturing solution L from the first accommodation section 22a to the third accommodation section 22c has been completed”. Next, the control unit 80 stops the bidirectional pump 40.

The comparison between the required time period and the actual elapsed time period, and the comparison between the pressure threshold value and the actual suction pressure, may be performed simultaneously. In this case, the control unit 80 stops the bidirectional pump 40 when either the actual elapsed time period coincides with the required time period or the actual suction pressure reaches the pressure threshold value. By promptly stopping the bidirectional pump 40 after the recovery of the culturing solution L to the storage tank 32 is completed as mentioned, an excessive load is prevented from acting on the bidirectional pump 40. The control unit 80 switches the recovery valve 66 to the closed state.

The culturing system 10 according to the present embodiment exhibits the following advantageous effects.

The culturing system 10 includes the culturing tank 20 configured to culture the microalgae and the supply device 30 configured to supply the culturing solution L to the culturing tank 20. The culturing tank 20 includes the plurality of accommodation sections 22 configured to accommodate the culturing solution L.

The supply device 30 includes the liquid delivery device 34 configured to deliver the culturing solution L to the plurality of accommodation sections 22, and the supply pipes 38 through which the culturing solution L delivered from the liquid delivery device 34 is delivered to the plurality of accommodation sections 22, respectively. The culturing system 10 includes the first float valves 48 configured to open and close the supply pipes 38. The first floats 50 constituting the respective first float valves 48 move upward in the depth direction X of the plurality of accommodation sections 22 as the liquid surfaces of the culturing solution L rise in the depth direction. When the first floats 50 moving upward abut against the first valve seats 52, the first float valves 48 close the supply pipes 38.

As described above, when the culturing solution L is supplied to each of the plurality of accommodation sections 22, the first float 50 moves upward as the liquid level of the culturing solution L rises. The supply of the culturing solution L in the accommodation section 22 is stopped when the supply pipe 38 is closed by the first float 50 moving upward. In this way, in accordance with the above configuration, the supply of the culturing solution L to the accommodation section 22 can be automatically stopped without providing a water level sensor for detecting the liquid level position of the culturing solution L. That is, in the culturing system 10, a water level sensor is not required. Accordingly, it is possible to reduce the cost of investment in equipment.

The liquid delivery device 34 also serves as the suction device 62 configured to recover the culturing solution L containing the microalgae from the plurality of accommodation sections 22. In this case, the configuration is simplified as compared with the culturing system in which the liquid delivery device 34 and the suction device 62 are individually provided.

Each of the plurality of accommodation sections 22 is made of a material exhibiting flexibility. The culturing system 10 includes the recovery pipes 68 and the second float valves 70 configured to close the recovery pipes 68. The second floats 72 constituting the respective second float valves 70 are positioned above the first floats 50 in the depth direction X.

In this configuration, when the suction device 62 is operated, the culturing solution L flows from the accommodation sections 22 into the recovery pipes 68. When the second floats 72 are pushed by the culturing solution L and rise, the second floats 72 are separated from the second valve seats 74, and the second float valves 70 are placed in the open state. Accordingly, the culturing solution L flows through the recovery pipes 68 and moves to the storage tank 32. Since each of the accommodation sections 22 is made of a material exhibiting flexibility, the accommodation sections 22 contract as the culturing solution L is recovered from the accommodation sections 22 and the inside of each of the accommodation sections 22 becomes negative pressure. The negative pressure keeps the first float valves 48 in the closed state. Accordingly, the culturing solution L flowing out to the recovery pipes 68 is prevented from returning to the accommodation sections 22 through the supply pipes 38.

Further, since the second float valves 70 are kept in the open state by the negative pressure, the culturing solution L can be easily recovered from all of the accommodation sections 22.

The supply device 30 includes the base pipe 36 connected to the liquid delivery device 34. The supply pipes 38 branch from the base pipe 36 and extend toward the plurality of accommodation sections 22, respectively.

In accordance with such a configuration, the culturing solution L can be supplied to all the accommodation sections 22 by using a single liquid delivery device 34. In the embodiment in which the liquid delivery device 34 also serves as the suction device 62, the culturing solution L can be recovered from all of the accommodation sections 22 by using the single liquid delivery device 34 (the suction device 62). In accordance with such a configuration, the cost of investment in equipment is reduced, as compared with the configuration in which the liquid delivery device 34 and the suction device 62 are individually connected to each of the plurality of accommodation sections 22.

The plurality of accommodation sections 22 are arranged from the upstream position to the downstream position in the flow direction (the direction of arrow A) of the culturing solution L in the base pipe 36 when the culturing solution L is supplied to the accommodation sections 22. The supply pipes 38 (the first supply pipe 38a to the third supply pipe 38c) respectively connecting the base pipe 36 and the plurality of accommodation sections 22 branch individually from the upstream position to the downstream position in the flow direction (the direction of arrow A) of the culturing solution L in the base pipe 36.

When the plurality of accommodation sections 22 are arranged in series as described above, a large amount of the culturing solution L per unit time tends to be supplied to the accommodation section 22 positioned relatively upstream as compared with the accommodation section 22 positioned relatively downstream. Accordingly, the accommodation of the predetermined amount of the culturing solution L is completed in the accommodation section 22 positioned relatively upstream before the accommodation section 22 positioned relatively downstream. Thereafter, the accommodation of the predetermined amount of the culturing solution L in the accommodation section 22 positioned relatively downstream is completed.

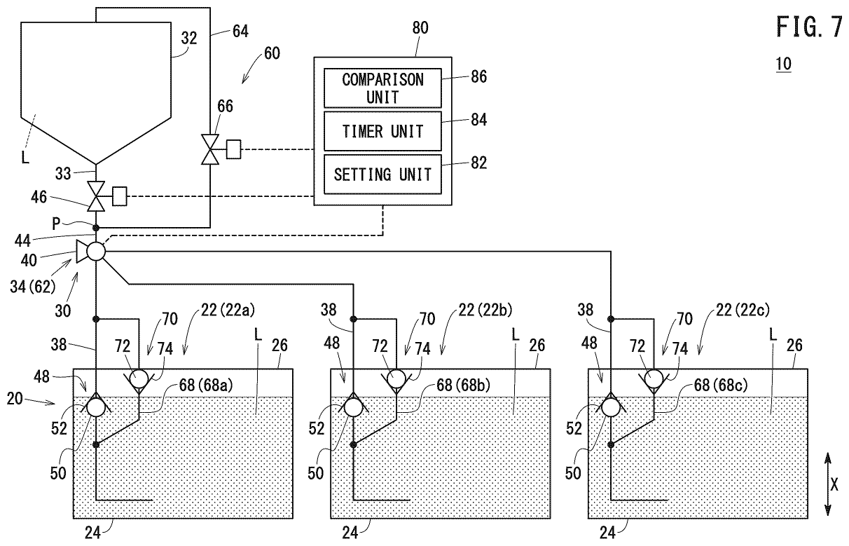

As shown in FIG. 7, in the case of a parallel arrangement in which the plurality of accommodation sections 22 are directly connected to the single liquid delivery device 34 through the supply pipes 38, since the culturing solution L is supplied from the single liquid delivery device 34 to each of the plurality of accommodation sections 22 at the same time, a large discharge pressure (supply pressure) is required. Accordingly, in such a case, it is necessary to use a large-sized liquid delivery device 34. In contrast, in the series arrangement shown in FIGS. 1 to 6, since the culturing solution L is sequentially supplied from the accommodation section 22 (first accommodation section 22a) positioned upstream to the accommodation section 22 (third accommodation section 22c) positioned downstream as described above, it is not necessary to use the large-sized liquid delivery device 34. Accordingly, it is possible to reduce the cost of investment in equipment. However, the present invention includes the embodiment shown in FIG. 7.

In the embodiment shown in FIG. 7, the number of the bidirectional pumps 40 is one. However, the number of the bidirectional pumps 40 may be the same as the number of the accommodation sections 22.

The culturing system 10 is provided with the control unit 80. The amounts of the culturing solution L accommodated in the respective plurality of accommodation sections 22 when the first floats 50 close the supply pipes 38 are defined as predetermined amounts of the culturing solution L, and the control unit 80 stops the liquid delivery device 34 when the control unit 80 has determined that all of the plurality of accommodation sections 22 accommodate the predetermined amounts of the culturing solution L, respectively.

In this way, when the control unit 80 has determined that all the first float valves 48 are in the closed state, the control unit 80 stops the liquid delivery device 34. This prevents an excessive load from being applied to the liquid delivery device 34.

For example, the required time period from when supply of the culturing solution L by the liquid delivery device 34 is started until when all of the plurality of accommodation sections 22 accommodate the predetermined amounts of the culturing solution L, respectively, is set in advance in the setting unit 82 of the control unit 80. The comparison unit 86 of the control unit 80 stops the liquid delivery device 34 when the actual elapsed time period from when the supply of the culturing solution L by the liquid delivery device 34 is started coincides with the required time period.

Alternatively, the control unit 80 acquires information on the actual discharge pressure of the culturing solution L in the liquid delivery device 34. The control unit 80 stops the liquid delivery device 34 when the actual discharge pressure has increased to the pressure threshold value set in advance in the setting unit 82.

In accordance with either or both of the above-mentioned ways, the stop timing of the liquid delivery device 34 can be set without using the water level sensor. Since the water level sensor is not required, the cost of investment in equipment for the culturing system 10 can be reduced.

The volumes of the plurality of accommodation sections 22 are each substantially the same. Each of the first floats 50 is disposed at the same position in the depth direction X of each of the plurality of accommodation sections 22.

In accordance with such a configuration, it is possible to supply substantially the same amount of the culturing solution L to each of the plurality of accommodation sections 22. Therefore, the amounts of the culturing solution L stored in the plurality of accommodation sections 22 can be uniform.

With respect to the above disclosure, the following supplementary notes are disclosed.

### Supplementary Note 1

The culturing system (10) is provided with the culturing tank (20) configured to culture the microalgae, and the supply device (30) configured to supply the culturing solution (L) to the culturing tank. The culturing tank includes the plurality of accommodation sections (22) configured to accommodate the culturing solution.

The supply device includes the liquid delivery device (34) configured to deliver the culturing solution to the plurality of accommodation sections, and the supply pipes (38) through which the culturing solution delivered from the liquid delivery device is delivered to the plurality of accommodation sections, respectively. The culturing system further includes the first float valves (48) configured to open and close the supply pipes. The first floats (50) constituting the respective first float valves move upward in the depth direction (X) of the plurality of accommodation sections as liquid surfaces of the culturing solution rise in the depth direction. Based on this movement, the first float valves close the supply pipes.

With the above-described closure, the supply of the culturing solution to the accommodation sections is stopped. In this way, in accordance with the above configuration, the supply of the culturing solution can be automatically stopped. Therefore, a water level sensor for detecting the liquid level position of the culturing solution is not necessary. Accordingly, it is possible to reduce the cost of investment in equipment.

### Supplementary Note 2

In the culturing system according to Supplementary Note 1, the liquid delivery device may also serve as the suction device (62) configured to recover the culturing solution from the plurality of accommodation sections. Each of the plurality of accommodation sections may be made of a material exhibiting flexibility. The culturing system may further include the recovery pipes (68) and the second float valves (70) configured to close the recovery pipes, and the second floats (72) constituting the respective second float valves may be positioned above the first floats in the depth direction.

Since the liquid delivery device also serves as the suction device, the configuration is simplified as compared with the culturing system in which the liquid delivery device and the suction device are individually provided.

When the suction device is operated, the second floats are pushed and rise by the culturing solution flowing from the accommodation sections into the recovery pipes. As a result, the second float valves are opened. Since each of the accommodation sections is made of a material exhibiting flexibility, the accommodation sections contract as the culturing solution L is suctioned from the accommodation sections and the inside of each of the accommodation sections becomes negative pressure. Further, since the first float valves are kept in the closed state by the negative pressure, the culturing solution is prevented from returning to the accommodation sections through the supply pipes. Further, since the second float valves are kept in the open state by the negative pressure, the culturing solution can be easily recovered from all of the accommodation sections.

### Supplementary Note 3

In the culturing system according to Supplementary Note 1 or 2, the supply device may include the base pipe (36) connected to the liquid delivery device, and the supply pipes may branch from the base pipe and extend toward the plurality of accommodation sections, respectively.

In accordance with such a configuration, the culturing solution can be supplied to all the accommodation sections by using a single liquid delivery device. When the liquid delivery device also serves as the suction device, the culturing solution can be recovered from all the accommodation sections by using the single liquid delivery device. In this case, the cost of investment in equipment is reduced, as compared with the configuration in which the liquid delivery device and the suction device are individually connected to each of the plurality of accommodation sections.

### Supplementary Note 4

In the culturing system according to Supplementary Note 3, the plurality of accommodation sections may be arranged from the upstream position to the downstream position in the flow direction of the culturing solution in the base pipe, and the supply pipes respectively connecting the base pipe and the plurality of accommodation sections may branch individually from the upstream position to the downstream position in the flow direction in the base pipe.

When the plurality of accommodation sections are individually connected to a single liquid delivery device, the liquid delivery device needs to output a large supply pressure (discharge pressure). Accordingly, in such a case, it is necessary to use a large-sized liquid delivery device. In contrast, in the above-described arrangement (series arrangement), since the culturing solution is sequentially supplied from the accommodation section positioned upstream to the accommodation section positioned downstream, it is not necessary to use the large-sized liquid delivery device. Accordingly, it is possible to reduce the cost of investment in equipment.

### Supplementary Note 5

In the culturing system according to any one of Supplementary Notes 1 to 4, the culturing system may further include the control unit (80), the amounts of the culturing solution accommodated in the respective plurality of accommodation sections when the first floats close the supply pipes are defined as predetermined amounts of the culturing solution, and the control unit may stop the liquid delivery device when the control unit has determined that all of the plurality of accommodation sections accommodate the predetermined amounts of the culturing solution, respectively.

When the control unit has determined that all the first float valves are in the closed state, the control unit stops the liquid delivery device. Thus, an excessive load is prevented from being applied to the liquid delivery device.

### Supplementary Note 6

In the culturing system according to Supplementary Note 5, the required time period from when supply of the culturing solution by the liquid delivery device is started until when all of the plurality of accommodation sections accommodate the predetermined amounts of the culturing solution, may be set in advance in the control unit, and the control unit may stop the liquid delivery device when the actual elapsed time period from when the supply of the culturing solution by the liquid delivery device is started coincides with the required time period.

In accordance with such a configuration, the stop timing of the liquid delivery device can be set without using a water level sensor. Since the water level sensor is not required, the cost of investment in equipment can be reduced.

### Supplementary Note 7

In the culturing system according to Supplementary Note 5 or 6, the control unit may acquire information on the supply pressure of the culturing solution in the liquid delivery device, and stop the liquid delivery device when the supply pressure increases to the pressure threshold value.

Also in this case, the water level sensor is not required as in the above case. Accordingly, it is possible to reduce the cost of investment in equipment.

### Supplementary Note 8

In the culturing system according to any one of Supplementary Notes 1 to 7, the volumes of the plurality of accommodation sections may be substantially the same as each other, and each of the first floats may be disposed at the same position in the depth direction of each of the plurality of accommodation sections.

In accordance with such a configuration, it is possible to supply substantially the same amount of the culturing solution to each of the plurality of accommodation sections. In other words, the amounts of the culturing solution L stored in the plurality of accommodation sections can be uniform.

Moreover, the present invention is not limited to the above-described disclosure, and various configurations can be adopted therein without departing from the essence and gist of the present invention.

What is claimed is:

1. A culturing system comprising:
a culturing tank configured to culture microalgae;
a supply device configured to supply a culturing solution to the culturing tank, wherein the culturing tank includes a plurality of accommodation sections configured to accommodate the culturing solution, and the supply device includes a liquid delivery device configured to deliver the culturing solution to the plurality of accommodation sections, and supply pipes through which the culturing solution delivered from the liquid delivery device is delivered to the plurality of accommodation sections, respectively; and
first float valves configured to open and close the supply pipes,
wherein first floats constituting the respective first float valves close the supply pipes by moving upward in a depth direction of the plurality of accommodation sections as liquid surfaces of the culturing solution rise in the depth direction.

2. The culturing system according to claim 1, wherein the liquid delivery device also serves as a suction device configured to recover the culturing solution from the plurality of accommodation sections,
each of the plurality of accommodation sections is made of a material exhibiting flexibility,
the culturing system further comprises recovery pipes and second float valves configured to close the recovery pipes, and
second floats constituting the respective second float valves are positioned above the first floats in the depth direction.

3. The culturing system according to claim 1, wherein the supply device includes a base pipe connected to the liquid delivery device, and the supply pipes branch from the base pipe and extend toward the plurality of accommodation sections, respectively.

4. The culturing system according to claim 3, wherein the plurality of accommodation sections are arranged from an upstream position to a downstream position in a flow direction of the culturing solution in the base pipe, and the supply pipes respectively connecting the base pipe and the plurality of accommodation sections branch individually from the upstream position to the downstream position in the flow direction in the base pipe.

5. The culturing system according to claim 1, further comprising one or more processors that execute computer-executable instructions stored in a memory,
wherein amounts of the culturing solution accommodated in the respective plurality of accommodation sections when the first floats close the supply pipes are defined as predetermined amounts of the culturing solution, and the one or more processors execute the instructions to cause the culturing system to stop the liquid delivery device when the one or more processors have determined that all of the plurality of accommodation sections accommodate the predetermined amounts of the culturing solution, respectively.

6. The culturing system according to claim 5, wherein a required time period from when supply of the culturing solution by the liquid delivery device is started until when all of the plurality of accommodation sections accommodate the predetermined amounts of the culturing solution is set in advance in the memory, and
the one or more processors execute the instructions to cause the culturing system to stop the liquid delivery device when an actual elapsed time period from when the supply of the culturing solution by the liquid delivery device is started coincides with the required time period.

7. The culturing system according to claim 5, wherein the one or more processors execute the instructions to cause the culturing system to acquire information on a supply pressure of the culturing solution in the liquid delivery device, and stop the liquid delivery device when the supply pressure increases to a pressure threshold value.

8. The culturing system according to claim 1, wherein volumes of the plurality of accommodation sections are substantially same as each other, and each of the first floats is disposed at a same position in the depth direction of each of the plurality of accommodation sections.
